(19) United States
(12) Patent Application Publication
(10) Pub. No.: US 2009/0155355 A1
(43) Pub. Date: Jun. 18, 2009

(54) Particles in a capsule

(75) Inventors: Marvin A. Heuer, Mississauga (CA); Jason Peters, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: Multi Formulations Ltd., Mississauga (CA)

(21) Appl. No.: 11/954,614

(22) Filed: Dec. 12, 2007

(51) Int. Cl. A61K 9/48; A61K 31/192; A61K 31/167; A61K 31/437

(52) U.S. Cl. 424/455; 514/570; 514/629; 514/29

(57) ABSTRACT

The invention relates to a capsule containing a heterogeneous mixture of active agents in the form of particles suspended in a liquid matrix and optionally active ingredients in the liquid matrix; the liquid matrix occupying less than the total internal volume of the capsule. The present invention further describes a capsule to allow the controlled-release of one or more active agents for specific desired benefits.

### FIELD OF THE INVENTION

The present invention relates to capsules for the delivery of active agents. Specifically, the active agents are contained within particles suspended in a liquid, which may also contain active agents.

### BACKGROUND OF THE INVENTION

The oral route of administration is, in general, the most convenient means of drug or active agent delivery. Oral dosage forms for administration of active ingredients, such as various drugs, include tablets, caplets and capsules. One of the main problems of oral administration using traditional technology is the rapid increase in plasma levels of the active agent. This may lead to problems of absorption and toxicity. Furthermore, when constant levels of an active agent are needed, repeated administrations are required. This limitation has largely led to the development of novel methods of controlling the release of active agents from oral dosage forms. Several methods are available to endow active agents in oral dosage forms with controlled release dissolution and include; physical and chemical modification, the use of specific excipients, and the use of specific coatings or encapsulation of either the dosage form itself or the active agents within the dosage form.

The encapsulation of active agents by various chemical reactions and within various matrices is an efficacious means of controlling the release of active agents (Majeti N. V. Ravi Kumar. Nano and Microparticles as Controlled Drug Delivery Devices. J Pharm Pharmaceut Sci. 3(2):234-258, 2000). This technology includes the coating of single molecules of an active agent up to multiporous beads that may contain many molecules of active agent and range in size from several nanometers up to about a millimeter in diameter.

Capsules are generally of two types—either hard-shelled or soft-shelled. Capsules have the advantage of being able to contain active agents in liquid form as solutions, emulsions or suspensions which allows for potentially improved bioavailability over solid dosage forms. Soft gelatin capsules have the further advantage of being easier to swallow than most other oral dosage forms.

The ability to suspend an active agent as encapsulated particles within a liquid and subsequently within a capsule offers numerous iterations for the controlled release oral administration of one or more active agents in a format that is flexible and easily administered.

### SUMMARY OF THE INVENTION

The foregoing needs and other needs and objectives that will become apparent for the following description are achieved in the present invention, which comprises a capsule having contained therein a heterogeneous mixture comprising a liquid and a plurality of particles. The particles comprise an active agent and are insoluble and freely movable within the liquid. The liquid and particle mixture occupy less than the total internal volume of the capsule; the remainder of the total internal volume is occupied by a bubble which is also freely movable in the liquid.

In another embodiment of the present invention, the liquid in which the particles are contained also comprises an active agent in solution. The active agent may be the same as, or distinct from, the active agent comprising the particles.

Additional embodiments of the present invention comprise particles providing for the controlled-release of an active agent.

Further embodiments of the present invention may comprise additional particles of a different subtype to allow for a complex release profile of an active agent contained therein.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

A used herein, the term “active agent” includes dietary supplements, diet supplements, nutritional supplements, supplemental compositions and supplemental dietary compositions or those similarly envisioned by those of skill in the art. Furthermore, “active agent” as disclosed herein belongs to category of compositions having at least one physiological function when administered to a mammal by conventional routes of administration.

Alternatively, formulations and nutritional compositions belonging to the present invention may be considered to be nutraceuticals. As used herein, the term “nutraceutical” is recognized and used in the art to describe a specific chemical compound or combination of compounds found in, organic matter for example, which may prevent, ameliorate or otherwise confer benefits against an undesirable condition. As is known in the art, the term “nutraceutical” is used to refer any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, soft-gel™ caplets, gel-caps and the like, not associated with food.

Extracts suitable for use in the present invention may be produced by extraction methods as are known and accepted in the art such as alcoholic extraction, aqueous extractions, carbon dioxide extractions, for example.

Examples of nutraceuticals include but are not limited to: alpha lipoic acid, various amino acids, and derivatives of amino acids, creatine, derivatives of creatine, caffeine, Coleus forskhlii extract, Camellia sinensis extract, conjugated linoleic acid, Evodia ruticarpa powder extract, melatonin, gamma-butyrobetaine, Geum japonicum extract, Hops extract, Leucojum aestivum extract, various minerals, picamilon, yohimbine and various vitamins.

Further those referred to as “active agents”, are commonly used pharmaceutical interventions such as various medicaments and over-the-counter (OTC) medicines or drugs. OTCs are available for the treatment of a number of ailments including pain, allergies, congestion and colds. Examples of such medicaments include but are not limited to: acetaminophen, Tylenol™, ibuprofen, acetylsalicylic acid, Aspirin™, pseudoephedrine, loratadine, dextromethorphan and diphenhydramine.

As used herein, the term “capsule” refers to a either a rigid, hard shell or a soft, pliable container that serves as a vehicle for liquids or semi-solids such as gels. Most capsules are made form gelatin derived from hydrolyzed animal collagen but as used herein, non-animal sources are also included. Capsules are often formed from two separate halves sealed together, but alternatively may be a one-piece form that is filled by injection and subsequently sealed. As used herein, the term “capsule” includes commonly used terms such as Gelcaps™, Softgel™ and Soft-gel™.

As used herein, the term or derivatives of the term “particle” refers to active agents suspended within a liquid which is encased or surrounded by a coating. The term or derivatives of the term “particle” as used herein is used to define minimally-sized entities such as molecules of active agents coated with, or reversibly entrapped within, a minimal number of compounds to result in the desired stability or dissolution properties for the active agent, often termed “microencapsulation”, up to substantially larger entities that may form a hollow substantially spherically-shaped vessel which may contain many molecules of active agent, often termed “beads” or “beadlets”. It is understood that the term or derivatives of the term “particle” includes common terms such as nanoparticles, microparticles, nanospheres, microspheres, beads and beadlets. It is further understood that, as the particles are in suspension in a liquid, the particles are stable in the liquid and that at least the outermost layer of the particle is not soluble or permeable to the liquid in which they are suspended. The particles coating or entrapping active agents are herein considered to be comprised of one or more excipients in addition to active agents. Such excipients typically form polymers under specific conditions to facilitate active agent trapping. Examples of particle-forming excipients include but are not limited to: polystyrene, cellulose propionate, poly(ethylene oxide)-poly(L-lactic acid)/poly(β-benzyl-L-aspartate), poly(lactide-co-glycolide)-[(propylene oxide)-poly(ethylene oxide)], polyphosphazene derivatives, polyethylene glycol, chitosan, chitosan-poly(ethylene oxide), alginate, alginate-poly-L-lysine, gelatin and gellan gum.

As used herein, the term “liquid” refers to material in a form of matter which moves freely and assumes the shape of the container within which it resides. The term “liquid” refers to any non-solid and non-gas and is understood to include aqueous and non-aqueous liquids such as organic liquids and oils. Liquids commonly used in the filling of capsules are those known not to react with components of a specific capsule shell and include, but are not limited to: castor oil, cottonseed oil, corn oil, olive oil, peanut oil, sesame oil, soybean oil, sunflower oil, caprylic acid, capric acid, glycerin, polyethylene glycol, propylene carbonate, triacetin and water. Furthermore, the liquid may be comprised of more than one liquid with excipients in order to facilitate the desired solubility characteristics.

As used herein, the term “complex release profile” refers to the release of active agents and is understood to be the actual or predicted release profile for a given active agent that is resultant from the summation of various distinct individual release profiles present. Several different release profiles are commonly known in the art and are described below.

“Unmodified-release” is understood to be defined as pertaining to the dissolution and bioavailability profile of an ingested dietary ingredient wherein no additional modifications, be it chemical or physical, have been made to the ingredient with the specific intent to alter the dissolution or bioavailability profile from that of ingredient in a naturally occurring form. It is also understood that unmodified-release is, essentially, immediate-release of active ingredients. This is further understood to be traditional- or conventional-release format where no slow-, delayed- or extended-release effect is incorporated.

“Quick-release” format is understood to be defined essentially as “unmodified-release”, as above. However, the term “quick-release” may further include components having modifications, chemical or physical, to enhance the rate of dissolution or bioavailability of active ingredients.

“Controlled-release” format is understood to be defined as a formulation of active ingredients and appropriate excipients in a specific format to facilitate a controlled- or non-immediate-release of active ingredients. The components of a controlled-release format may have been subjected to additional modifications, be it chemical or physical, with the specific intent to alter the dissolution or bioavailability profile from that of ingredient in a naturally occurring form. Examples of controlled-release profiles include delayed-release, slow-release, sustained-release, extended-release and time- or timed-release.

During the production of liquid-filled capsules, particularly with regard to large-scale automated production, numerous imperfections can manifest. These perfections include stains or other discolorations, incorrect size, shape defects such as bulges, and in inclusion of bubbles. The presence of bubbles, consisting of air trapped during the manufacturing process, may be problematic in cases where the active agents contained in the capsule are susceptible to oxidative degradation. However, in cases where the active agents are not susceptible to being rendered inert or toxic by the presence of air, the bubble may remain as is. In various embodiments of the present invention, the air in the bubble may replaced by nitrogen, or another inert gas, to prevent the oxidation of active agents contained in the capsule. Furthermore, the capsules may be produced in a nitrogen, or other inert gas, environment to avoid the need to replace the contents of the bubble.

In certain situations, it is advantageous to have a bubble present in a liquid-filled capsule. In such situations, a bubble will facilitate mixing of the contents of the capsule. This is of particular interest with respect to the capsules of the present invention in which particles comprising an active agent are dispersed within a liquid, which, in certain embodiments, also comprises an active agent. Particles may be susceptible to agglomeration, therefore decreasing the surface area-to-volume ratio of the active particles, which may hamper intended dissolution characteristics. Also, if more than one type of particle is present in the capsule, the different particles may segregate based on differential densities of the particles, which may also negatively affect dissolution. The movement of the bubble within the liquid will act to ensure that the particles remain with a high degree of entropy.

The mixing of liquids of small volume is not subject to the same considerations as is the mixing of large volumes. In micro- or mini systems it is more difficult to generate turbulence as is commonly done during mixing of larger volumes (Liu R H, Yang J, Pindera M Z, Athavale M, Grodzinski P. Bubble-induced acoustic micromixing. Lab Chip. August 2002;2(3):151-7). The primary force responsible for small-scale mixing is diffusion, which is typically inefficient. However, the movement of bubbles through a liquid is also known to generate mixing forces (Darmana, D., Deen, N. G. and Kuipers, J. A. M. “Detailed Modeling of Hydrodynamics, Mass transfer and Chemical Reactions in a Bubble Column using a Discrete Bubble Model”, Chem. Eng. Sci. 2005 Vol. 60, No. 12, pp. 3383-3404).

It is herein understood by the inventors that the presence of a bubble in a liquid comprising particles contained within a capsule will facilitate mixing of the particles in the liquid by movement of the bubble throughout the liquid, thus preventing agglomeration of the particles and maintaining the desired dissolution characteristics of the capsule and the active agents contained therein.

The preferred embodiment of the present invention comprises a capsule having contained therein a heterogeneous mixture comprising a liquid and a plurality of particles wherein the particles comprise an active agent and are insoluble and freely movable within the liquid, the liquid and particle mixture filling less than the total internal volume of the capsule wherein the remainder of the total internal volume is occupied by a bubble which is freely movable in the liquid.

In another embodiment of the present invention, the liquid contains an active agent in addition to the insoluble particles described supra. The active agent may be the same as, or distinct from, the active agent comprising the particles and depending on the properties of the active agent, may form a solution, an emulsion or a suspension in the liquid.

Additional embodiments of the present invention comprise particles providing for the controlled-release of an active agent.

Further embodiments of the present invention comprise particles of different subtypes to allow for a complex release profile of an active agent contained therein.

Specific embodiments of the present invention are directed at methods and compositions for treating pain. Such embodiments utilize active agents known to reduce pain-associated symptoms. Such pain-associated symptoms include but are not limited to muscle aches, head aches, muscle or joint tenderness, tissue inflammation and fever. Some commonly used active agents are further discussed below.

Ibuprofen

Ibuprofen belongs to the class of actives known as non-steroidal anti-inflammatory drugs (NSAID) and is often used to treat pain, particularly pain involving inflammation. Typically, OTC ibuprofen is available in 200 mg doses to be taken every 4 hours, not to exceed 1200 mg per day. The maximum plasma concentration of oral ibuprofen in humans is reached in about 1.3 hrs following a single 400 mg dose (Canaparo R, Muntoni E, Zara G P, Della Pepa C, Berno E, Costa M, Eandi M. Determination of Ibuprofen in human plasma by high-performance liquid chromatography: validation and application in pharmacokinetic study. Biomed Chromatogr. June 2000;14(4):219-26) and has a half-life of about 2 hrs (Trappe T A, White F, Lambert C P, Cesar D, Hellerstein M, Evans W J. Effect of ibuprofen and acetaminophen on postexercise muscle protein synthesis. Am J Physiol Endocrinol Metab. March 2002;282(3):E551-6).

The bioavailability of various forms or ibuprofen has been demonstrated in humans (Tamilvanan S, Sa B. In vitro and in vivo evaluation of single-unit commercial conventional tablet and sustained-release capsules compared with multiple-unit polystyrene microparticle dosage forms of Ibuprofen. AAPS PharmSciTech. Sep. 1, 2006;7(3):72). Conventional ibuprofen tablets were found to give peak plasma levels at about 2 hrs, steadily decreasing up to about 6 hrs; commercial sustained-release capsules were found to peak at about 6 hrs and decrease to about 24 hrs; while ibuprofen-loaded polystyrene particles with diameter of 275 82 m were found to peak at about 4 hrs and decrease to about 24 hrs. Both the commercial sustained release capsules and the microparticles maintained a minimum effective dose over 24 hrs.

Certain embodiments of the present invention comprise ibuprofen. In some embodiments of the present invention, the ibuprofen is provided as particles suspended within a liquid. In further embodiments, the ibuprofen is provided as controlled-release particles suspended within a liquid. A single serving of capsules of the present invention may comprise from about 10 mg to about 400 mg of ibuprofen per serving.

Acetaminophen

Acetaminophen is an effective OTC analgesic but lacks significant anti-inflammatory properties. The maximum OTC dose of acetaminophen is considered to be about 4000 mg per day. Acetaminophen has similar pharmacokinetic properties to ibuprofen, with peak plasma levels being observed within 0.5-2 hrs and a plasma half-life of about 2 hrs (Trappe T A, White F, Lambert C P, Cesar D, Hellerstein M, Evans W J. Effect of ibuprofen and acetaminophen on postexercise muscle protein synthesis. Am J Physiol Endocrinol Metab. March 2002;282(3):E551-6).

Certain embodiments of the present invention comprise acetaminophen. In some embodiments of the present invention, the acetaminophen is provided as particles suspended within a liquid. In further embodiments, the acetaminophen is provided as controlled-release particles suspended within a liquid. A single serving of capsules of the present invention may comprise from about 25 mg to about 500 mg of acetaminophen per serving.

Caffeine

Caffeine is a plant alkaloid having stimulant effects in humans. Caffeine is a widely consumed substance, being a natural component of popular beverages such as coffee and tea but further added to many other beverages such as soda pop and energy drinks. It is estimated that the average worldwide consumption of caffeine is about 70 mg per day per person (Donovan J L, DeVane C L. A primer on caffeine pharmacology and its drug interactions in clinical psychopharmacology. Psychopharmacol Bull. 2001 Summer;35(3):30-48). Caffeine is also contained in many OTC and prescription drugs. The half-life of caffeine is highly variable but the mean time is about 5 hrs with peak levels being reached after about 1 hr (Carregaro A B, Woods W E, Tobin T, Queiroz-Neto A. Comparison of the quantification of caffeine in human plasma by gas chromatography and ELISA. Braz J Med Biol Res. June 2001;34(6):821-4).

Caffeine has been shown to increase the effects of analgesics (Lipton R B, Stewart W F, Ryan R E Jr, Saper J, Silberstein S, Sheftell F. Efficacy and safety of acetaminophen, aspirin, and caffeine in alleviating migraine headache pain: three double-blind, randomized, placebo-controlled trials. Arch Neurol. February 1998;55(2):210-7). Caffeine is commonly used as an analgesic adjuvant, particularly in formulations with acetaminophen (Zheng Q S, Wang X W, Gui C Q, Sun R Y. Quantitative design of optimal analgesic combination of acetaminophen, caffeine, and butalbital. Acta Pharmacol Sin. August 2001;22(8):691-6) and has been shown to enhance and prolong the effects of acetaminophen by accelerating the absorption of acetaminophen. Caffeine has also been shown to enhance the analgesic effects of ibuprofen in humans.

Certain embodiments of the present invention comprise caffeine or derivatives of caffeine. Preferably, the caffeine or derivative of caffeine is provided in the liquid within the capsule of the present invention in various embodiments. A single serving of capsules of the present invention may contain from about 1 mg to about 400 mg of caffeine or derivatives of caffeine.

Additional embodiments of the present invention comprise caffeine in quick-release particles. Some such embodiments may further comprise an additional active agent wherein the additional active agent is in controlled-release particles. In specific embodiments of this type, the additional active agent to be provided in controlled-release particles has an unmodified half-life of a shorter duration than the half-life of caffeine such that the effective half-life resulting for the controlled-release particles is closer to the half-life of caffeine. These embodiments are particularly advantageous in specific cases wherein the additional agent is in any way known to synergize with, cooperate with or benefit from caffeine.

Embodiments of the present invention may employ particle-milling technology for enhanced utility and efficacy. U.S. patent application Ser. No. 11/709,526 entitled “Method For Increasing The Rate And Consistency Of Bioavailability Of Supplemental Dietary Ingredients” filed Feb. 21, 2007, which is herein fully incorporated by reference discloses the use of particle-milling for the purposes of increasing the rate of bioavailability following oral administration of components comprising supplemental dietary compositions. The increased bioavailability of a compound or ingredients is achieved via a reduction in particle size using a “fine-milling” technique. For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role. The components of the present invention may fine-milled in order to quicken the rate of dissolution.

Additionally, U.S. patent application Ser. No. 11/709,525 entitled “Method for a Supplemental Dietary Composition Having a Multi-Phase Dissolution Profile” filed Feb. 21, 2007, also herein fully incorporated by reference, discloses that components of the present invention may be used as portions of both non-milled and fine-milled, in order to provide a bi-phasic dissolution profile. Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent. Further to such additional embodiments, the components of the present invention may be present in portions fine-milled to varying degrees thereby providing a multi-phasic dissolution profile as is disclosed in the preceding application reference.

Furthermore, the dosage form of the capsule may be provided in accordance with customary processing techniques for herbal and nutritional supplements in any of the forms mentioned above. Additionally, the capsule set forth in the example embodiment herein may contain any appropriate number and type of excipients, as is well known in the art.

Although the following examples illustrate the practice of the present invention in three of its embodiments, the examples should not be construed as limiting the scope of the invention. Other embodiments will be apparent to one of skill in the art from consideration of the specifications and example.

### EXAMPLES

### Example 1

A supplement is provided in soft capsules for administration to individuals wishing to relieve painful conditions such as headache or muscle-ache. The liquid in the capsule is comprised of sesame oil. Each serving of the supplement contains the following:
about 225 mg of caffeine (in liquid), about 200 mg of sustained-release ibuprofen particles (sustained-release over 5-hrs).

A serving of the soft capsules are to be taken with water at the onset of pain symptoms and continued every 4 to 6 hours, as long as pain lasts.

### Example 2

A supplement is provided in soft capsules for administration to individuals wishing to relieve painful conditions such as headache or muscle-ache. The liquid in the capsule is comprised of soybean oil and glycerin. Each serving of the supplement contains the following:
about 100 mg of caffeine (in liquid), about 100 mg of ibuprofen (in liquid), about 100 mg of sustained-release ibuprofen particles (sustained-release over 12-hrs) and about 250 mg of caffeine (sustained-release over 10-hrs).

A serving of the soft capsules are to be taken with water at the onset of pain symptoms and continued once or twice daily until pain subsides.

### Example 3

A supplement is provided in hard capsules for administration to individuals wishing to maximize energy levels for intense exercise sessions. The liquid in the capsule is comprised of sesame oil. Each serving of the supplement contains the following:
about 100 mg of caffeine (in liquid) and a vitamin blend (quick-release particles containing about 400 mg vitamin C, about 50 mg thiamin, about 50 mg niacin, about 50 mg vitamin B6 and about 0.1 mg vitamin B12).

A serving of the soft capsules are to be taken with water 30 to 60 minutes prior to exercise.

### Extensions and Alternatives

In the foregoing specification, the invention has been described with a specific embodiment thereof; however, it will be evident that various modifications and changes may be made thereto without departing from the broader spirit and scope of the invention.

What is claimed:

1. A capsule having contained therein a heterogeneous mixture comprising a liquid and a plurality of particles wherein the particles comprise an active agent;
the plurality of particles are insoluble and freely movable within the liquid;
wherein the liquid and plurality of particles occupy less than the total internal volume of the capsule;
a freely movable bubble occupying the remainder of the internal volume of the capsule.

2. The freely movable bubble of claim 1, wherein movement of the freely movable bubble causes perturbation of the liquid and the particles contained within the capsule.

3. The capsule of claim 1, wherein the particles provide controlled-release of the active agent.

4. The capsule of claim 3, wherein the particles are of different subtypes, each subtype having a distinct and different release profile from the other subtypes, wherein the combination of different release profiles provides a complex release profile of the active agent.

5. The capsule of claim 1, wherein the active agent is selected from the group consisting of acetaminophen, ibuprofen, aspirin, pseudoephedrine, loratadine, dextromethorphan and diphenhydramine.

6. The capsule of claim 1, wherein the liquid comprises an active agent.

7. The capsule of claim 6, wherein the active agent in the liquid is caffeine or derivative of caffeine.
